(19) United States
(12) Patent Application Publication
(10) Pub. No.: US 2015/0112121 A1
(43) Pub. Date: Apr. 23, 2015

(54) PACIFIER SYSTEM ADAPTED TO IMITATE THE CLOSENESS OF A SUBJECT AT REST

(71) Applicant: RORO ehf, Reykjavik (IS)

(72) Inventor: Eggertsdottir Eyrun, Reykjavik (IS)

(73) Assignee: RORO ehf., Reykjavik (IS)

(21) Appl. No.: 14/394,977

(22) PCT Filed: Apr. 16, 2013
(86) PCT No.: PCT/IS2013/000001

§ 371 Date: Oct. 16, 2014

(30) Foreign Application Priority Data
Apr. 16, 2012 (IS) 8997

(51) Int. Cl. A61M 21/02; A63H 3/00

(52) U.S. Cl. CPC A61M 21/02; A63H 3/003; A61M 2021/0027
USPC 600/28

(57) ABSTRACT

This invention relates to a pacifier system adapted to imitate the closeness of a subject at rest. A pacifier soft body such as a doll having a cavity therein is provided and a playback device adapted to be fitted into the cavity, where the playback device is operable to play a recording of the subject at rest and thus imitate the closeness of the subject

### FIELD OF THE INVENTION

The present invention relates to a pacifier system and a pacifier system assembly adapted to imitate the closeness of a subject at rest.

### BACKGROUND OF THE INVENTION

Several soft toys with electrical devices inside that are meant for newborns and small children having been developed in the last years. They are often referred to as audio pacifiers (e.g. Sleep Sheep, Gentle Giraffe, Ewan the Dream Sheep, Back to Sleep Bear). Most of them play different white noises for a short time and some try to imitate the womb experience. These devices do not provide continuous playing of sounds and do not offer the sound of real life. Further to this, they are not shaped to hold the child and most are not meant for holding at all and typically do not have human characteristics but look like animals. Accordingly, none of the above mentioned toys are suitable for implementation at e.g. hospitals or at home for replacing at least up to some extend the missing hug or holding of a parent near the chest. As an example, many hospitals and NICUs either have limited or no space at all for parents to hold their children for long hours. Also the smallest and most fragile premature children cannot come out of their incubators and other only for a short period of time. It is especially important to make up for the isolation and lack of closeness that these premature children have to suffer. This lack of closeness may even result in the chance of apnea episodes.

The inventor of the present invention has appreciated that there is a need for a mechanism that imitates the closeness of parent at rest and has in consequence devised the present invention.

### SUMMARY OF THE INVENTION

It would be advantageous to achieve a pacifier system that is capable of stabilizes heartbeat and breathing in infants and premature children and reduces the chance of apnea episodes. In general, the invention preferably seeks to mitigate, alleviate or eliminate one or more of the above mentioned disadvantages singly or in any combination. In particular, it may be seen as an object of the present invention to solves the above mentioned problems, or other problems, of the prior art.

To better address one or more of these concerns, in a first aspect of the invention a pacifier system is provided adapted to imitate the closeness of a subject at rest, comprising:

a pacifier soft body having an cavity therein,

a playback device adapted to be fitted into said cavity,

wherein the playback device is operable to play a recording of said subject at rest and thus imitate the closeness of the subject. Accordingly, said pacifier system may be used for stabilizing the heartbeat and breathing in infants and premature children and thus reduce the chance of apnea episodes but apnea is thought to be the leading cause of Sudden Infant Death Syndrome (SIDS).

In one embodiment, said pacifier soft body comprises an opening means for providing an access into said cavity so as to providing installing or removing feature said playback device. In one embodiment, said opening means is an openable and closable pocket. In that way, the playback device may easily be installed or removed from the doll.

In another embodiment, said playback device further comprises a recording function. Accordingly, if the subject is a parent it can easily record the sound of the heartbeat and breathing and other sounds that the infant or premature child knows. The playback device may as an example be, but is not limited to, a MP3 player or a similar player that is compact and provides such playback and recording feature.

In still another embodiment, said playback device further comprises adjustable volume function and a timer for setting the playback time.

In yet another embodiment, said subject is a parent and wherein the play recording is heartbeat and breathing of said parent at rest. Accordingly, by playing heartbeat and breathing of e.g. a mother at rest the sounds assimilates a hug or a hold of a parent, which has a positive effects on the child's sleep patterns and overall wellbeing of the child and parents.

Further to this, the accumulation of the above mentioned features provide an optimal animation of the closeness of a parent, namely the sounds of real life heartbeat and breathing of a mother, the ability to play continuously for many hours (can “sleep” with the child through the night), to emit audio waves that create small movements that the child can sense and the softness of natural fabric (that provides closeness and warmth and can transfer smell).

In one embodiment, the pacifier body is a doll. The doll may be provided with human facial characteristics that infants and small children enjoy and prefer to look at above all other things and has a shape that is adapted to the child to give a hug or a hold to the child, but the design of the doll may be based on multiple research that have shown the clear benefits of closeness of an adult human being, the closeness of the sound of breathing and/or heartbeat, the closeness of a soft object like a doll or a cloth of some kind (often referred to as “lovey”)

Further advantages of pacifier system include:

offering a solution for children and infants who are not able to enjoy the hold of their parents as with neonates and prematurely born children which are confined to incubators

stabilizing children's breathing and heartbeat

decreasing the risk of apnea among children

promoting continuous sleep among children

reducing the risk of problems associated with sleep disorders in children such as distress, crying, weight loss and sudden infant death syndrome (SIDS)

supporting and promotes children's neural development and growth

increasing the feeling of wellbeing for children

enabling children to adjust to sleeping alone

helping children adapt to different sleeping arrangements

helping children to soothe themselves

helping children to fall asleep by themselves

offering parents longer and more coherent sleep

reducing the risk of difficulties associated with sleep deprivation among parents such as depression, stress, fatigue, illness, accidents and strained communication with children and spouse

parents can be confident that their child receives closeness and intimacy in their absence

creating the sense of ease and security among parents

supporting and facilitating the work of health employees that care for young children.

In one embodiment, the play recording is selected from:

heartbeat of a person, or

breathing of a person, or

soothing sounds,

a combination of one or more of the above.

By the term soothing sound is e.g. meant relaxing acoustics such as pan flute or a relaxing music or environmental sound such as ocean waves and the like.

In one embodiment, said pacifier soft body is a selected from:

a pillow,

a hand-shaped pillow,

an animal.

According to a second aspect, the invention further relates to a pacifier system assembly, comprising:

a pacifier soft body having an cavity therein,

a playback device operable to play recording of said subject at rest and thus imitate the closeness of the subject adapted to be fitted into said cavity.

In general the various aspects of the invention may be combined and coupled in any way possible within the scope of the invention. These and other aspects, features and/or advantages of the invention will be apparent from and elucidated with reference to the embodiments described hereinafter.

### BRIEF DESCRIPTION OF THE DRAWINGS

Embodiments of the invention will be described, by way of example only, with reference to the drawings, in which

FIG. 1 shows a pacifier soft body containing a cavity that is made of soft material,

FIG. 2 shows a device adapted to fit into the cavity shown in FIG. 1.

### DESCRIPTION OF EMBODIMENTS

The hug or holding of a parent near the chest (as in “kangaroo care”) has been shown to have positive effects on infants and premature children. Considerable research throughout the world on the benefits of KC shows that KC promotes cardio-respiratory stabilization, improves thermoregulation, increases rate of infant weight gain, shortens hospital stays, functions as analgesia and reduces maternal stress levels. KC can also improve infant's oxygen saturation and significantly reduce their oxygen requirements during the contact time. Other noted effects are longer periods of quiet sleep, more weight gain, less crying and fewer negative emotional signs and improved neuorological development. Other research that study the effect of closeness, breathing and heartbeat show similar results. Kangaroo care or the holding of a child is not always possible. Many hospitals and NICUs either have limited or no space facilities for parents to hold their children for long hours. Also the smallest and most fragile premature children cannot come out of their incubators. It is especially important to make up for the isolation and lack of closeness that some premature children have to suffer.

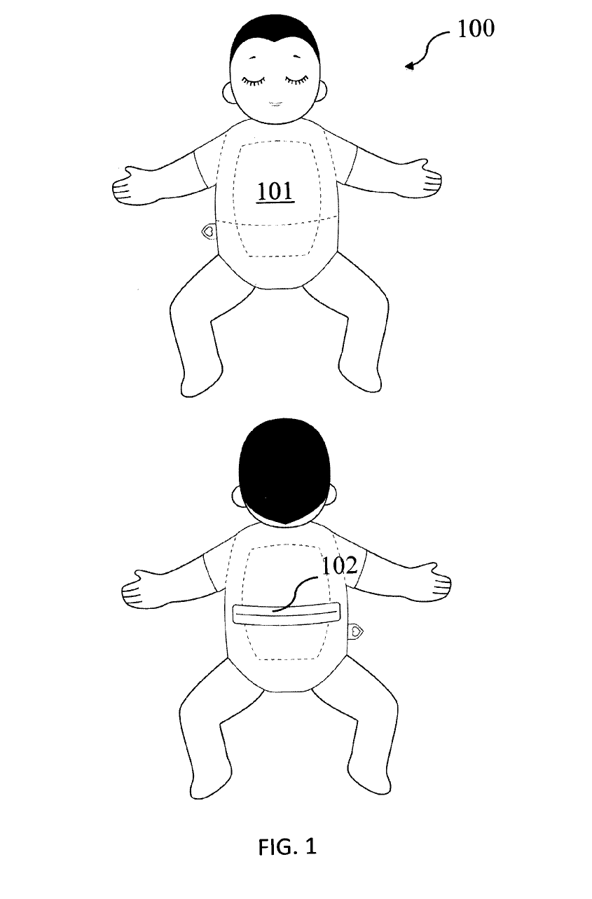

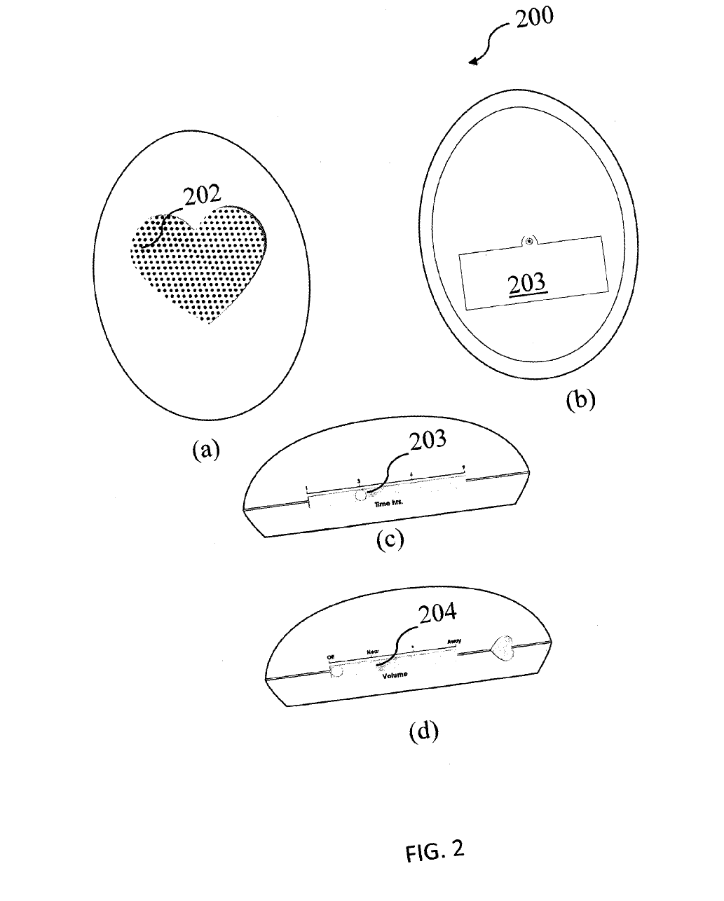

FIG. 1 shows a front side and a back side of a pacifier system according to the present invention comprising a pacifier soft body having cavity therein adapted to host a playback device (see FIG. 2). As depicted here, the pacifier system is a mechanical doll system 100 and the pacifier soft body is a doll. The pacifier soft body should of course not be construed as being limited to doll, but it may just as well be a pillow or any types of animal.

In this embodiment the doll is made of soft material that is preferable made of natural fabrics.

The dotted line inside the doll marks the cavity where a device, that will be discussed in more details in relation to FIG. 2, is fitted in. In the embodiment depicted here, the doll comprises an opening means 102 for providing an access into said cavity so as to providing installing or removing of said playback device, where the opening may e.g. be an openable and closable pocket.

FIG. 2 shows a playback device 200 according to the present invention adapted to be fitted inside cavity the doll shown in FIG. 1, where the fitting may be adapted to the desired positioning of the doll in relation to the infant. The playback device 200 comprises at least one speaker 202 arranged at one side of the device and a battery, such as a rechargeable battery 203, operable to play a recording of a subject at rest and thus imitate the closeness of the subject. In a preferred embodiment, the playback device comprises a recording function so as to enable a recording of said subject, which may be a parent, e.g. a mother. The playback may further comprise a timer 203 (see FIG. 2c) to adjust the time of the playback and a volume 204 (see FIG. 2d) to adjust the volume of the playback. The subject may be, but is not limited to, a parent and wherein the play recording is heartbeat and breathing of said parent at rest, e.g. the heartbeat and breathing of a mother at rest.

In one embodiment, the device 200 may be, but is not limited to, about 10 cm in length and 7.3 in width. As depicted here, the acoustics will be from one side if the device (and thus the doll), but the volume may e.g. only be selected in a small range that is suitable to an infants hearing when the doll is either near the infant or away from it (for example fastened to the crib).

Accordingly, the mechanical doll system depicted in FIGS. 1 and 2 includes the many important factors that closeness consists of, namely the sound of real life heartbeat and breathing of e.g. a mother (recorded simultaneously), the ability to play continuously for many hours (can “sleep” with the child through the night), and to emit audiowaves that create small movements that the child can sense. Further to this, the softness of natural fabrik of the doll may additionally provide additional closeness and warmth and may even transfer smell from the parent.

As depicted here, the doll comprise human facial characteristics (that infants and small children enjoy and prefer to look at above all other things) and is shaped to give a hug or a hold to the child.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage. Any reference signs in the claims should not be construed as limiting the scope.

1-10. (canceled)

11. A pacifier system adapted to imitate the closeness of a subject at rest, comprising:
a pacifier soft body having an cavity therein,
a playback device adapted to be fitted into said cavity, wherein the playback device is operable to play a recording of said subject at rest and thus imitate the closeness of the subject.

12. A pacifier system according to claim 11, wherein said playback device further comprises a recording function.

13. A pacifier system according to claim 11, wherein said pacifier soft body comprises an opening means for providing an access into said cavity so as to providing installing or removing feature said playback device.

14. A pacifier system according to claim 11, wherein said opening means is an openable and closable pocket.

15. A pacifier system according to claim 13, wherein said playback device further comprises a recording function.

16. A pacifier system according to claim 11, wherein said playback device further comprises adjustable volume function and a timer for setting the playback time.

17. A pacifier system according to claim 11, wherein said subject is a parent and wherein the play recording is heartbeat and breathing of said parent at rest.

18. A pacifier system according to claim 11, wherein the play recording is selected from:
heartbeat of a person, or
breathing of a person, or
soothing sounds,
a combination of one or more of the above.

19. A pacifier system according to claim 11, wherein said pacifier soft body is a doll.

20. A pacifier system according to claim 11, wherein said pacifier soft body is a selected from:
a pillow,
a hand-shaped pillow,
an animal.

21. A pacifier system assembly, comprising:
a pacifier soft body having an cavity therein,
a playback device operable to play recording of said subject at rest and thus imitate the closeness of the subject adapted to be fitted into said cavity.
